(19) United States
(12) Patent Application Publication
(10) Pub. No.: US 2025/0288279 A1
(43) Pub. Date: Sep. 18, 2025

(54) PUSH HANDLE OF ULTRASOUND DIAGNOSTIC APPARATUS

(71) Applicant: FUJIFILM Corporation, Tokyo (JP)

(72) Inventor: Naoyuki Naruse, Tokyo (JP)

(73) Assignee: FUJIFILM Corporation, Tokyo (JP)

(21) Appl. No.: 19/077,038

(22) Filed: Mar. 11, 2025

(30) Foreign Application Priority Data
Mar. 18, 2024 (JP) 2024-041856

(51) Int. Cl. A61B 8/00

(52) U.S. Cl. CPC A61B 8/4405

(57) ABSTRACT

A push handle of an ultrasound diagnostic apparatus includes a handle top that includes grip portions, and a handle support of which an upper end is fixed to the handle top and a lower end is fixed to an apparatus body. The handle support includes an upper support member that is integrated with the handle top, a lower support member that is provided on the apparatus body, and an intermediate support member of which an upper end portion is attachably and detachably coupled to the upper support member and a lower end portion is attachably and detachably coupled to the lower support member. The upper support member and the lower support member can be directly coupled to each other without the intermediate support member therebetween. In a state where the upper support member and the lower support member are coupled to each other via the intermediate support member, a height of the push handle is increased. In a state where the upper support member and the lower support member are directly coupled to each other, the height of the push handle is reduced.

### CROSS-REFERENCE TO RELATED APPLICATION

This application claims the priority benefit of Japan application serial no. 2024-041856, filed on Mar. 18, 2024. The entirety of the above-mentioned patent application is hereby incorporated by reference herein and made a part of this specification.

### BACKGROUND OF THE INVENTION

### 1. Field of the Invention

The present invention relates to a structure of a push handle of an ultrasound diagnostic apparatus.

### 2. Description of the Related Art

An ultrasound diagnostic apparatus transmits and receives ultrasound to and from the inside of a body of a patient to acquire an ultrasound tomographic image or the like, and displays the acquired image on a monitor. Such an ultrasound diagnostic apparatus is used in many hospitals. The ultrasound diagnostic apparatus is generally used in a state of being fixed in an examining room, but may be moved to another examining room or another hospital room to be used. For this reason, a general ultrasound diagnostic apparatus is adapted to include casters on the bottom thereof and to be capable of being moved in a case where a user pushes or pulls a handle provided on an apparatus body. JP2009-273507A discloses an ultrasound diagnostic apparatus of which a handle can be raised and lowered according to a height of a worker.

### SUMMARY OF THE INVENTION

A simpler structure that allows a height of a handle top of a handle used to move the ultrasound diagnostic apparatus to be changed is required.

A push handle of an ultrasound diagnostic apparatus according to an aspect of the present invention includes a handle top that extends in a left-right direction and a handle support of which an upper end is fixed to the handle top and a lower end is fixed to an apparatus body, and is installed upright on the apparatus body. The handle support includes an upper support member that is integrated with the handle top, a lower support member that is provided on the apparatus body, and an intermediate member of which an upper end portion is attachably and detachably coupled to the upper support member and a lower end portion is attachably and detachably coupled to the lower support member. The upper support member and the lower support member can be directly coupled to each other without the intermediate member therebetween.

In a case where the upper support member and the lower support member are coupled to each other via the intermediate member, a height of the push handle is increased. In a case where the upper support member and the lower support member are directly coupled to each other without using the intermediate member, the height of the push handle is reduced.

The intermediate member may be bent. In a case where the intermediate member is interposed between the upper support member and the lower support member, the handle top is positioned rearward as compared to a case where the intermediate member is not interposed. Accordingly, interference between the push handle and other components of the ultrasound diagnostic apparatus, particularly the articulated arm supporting the main monitor is prevented.

The push handle may include two handle supports, and the two handle supports are disposed to be separated from each other in the left-right direction. The push handle may include a lateral member that connects right and left intermediate members. Since the two handle supports are connected to each other by the lateral member, the stiffness of the push handle, particularly stiffness against torsion, is improved.

Coupling portions of the upper support member and the intermediate member and coupling portions of the lower support member and the intermediate member may have a halved shape. In a case where the upper support member and the intermediate member are coupled to each other and the lower support member and the intermediate member are coupled to each other, an outer shape of the coupling portion and an outer shape of a portion adjacent to the coupling portion may be identical to each other. At a coupled portion between the members, a step is not formed on the surface of the handle support.

Since the height of the push handle is changed, the height of the push handle can be set to heights where a tall worker and a short worker easily use the push handle in work in which a worker moves the ultrasound diagnostic apparatus while holding the push handle.

### BRIEF DESCRIPTION OF THE DRAWINGS

FIG. 1 is a front perspective view showing an appearance of an ultrasound diagnostic apparatus.

FIG. 2 is a rear perspective view showing the appearance of the ultrasound diagnostic apparatus and is a diagram showing a state where a height of a push handle is increased.

FIG. 3 is a rear perspective view showing the appearance of the ultrasound diagnostic apparatus and is a diagram showing a state where the height of the push handle is reduced.

FIG. 4 is a side view of the ultrasound diagnostic apparatus and is a diagram showing the disposition of the push handles having different heights.

FIGS. 5A and 5B are perspective views showing a structure of the push handle in detail, in which FIG. 5A shows an assembled state and FIG. 5B shows a disassembled state.

FIG. 6 is a diagram showing a cross section of the push handle, particularly a cross section of a handle support.

### DESCRIPTION OF THE PREFERRED EMBODIMENTS

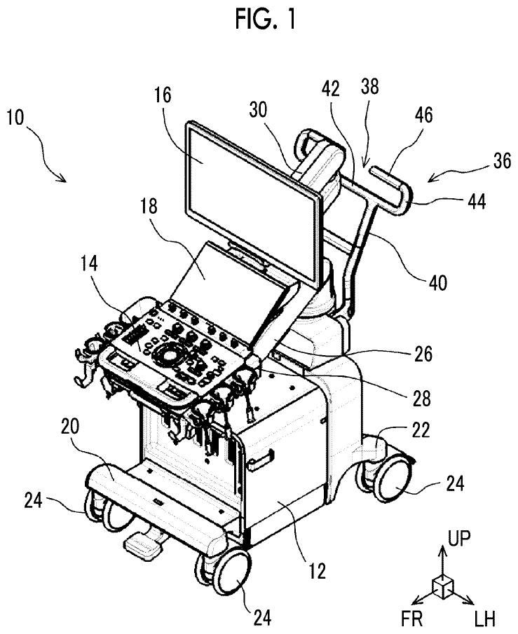

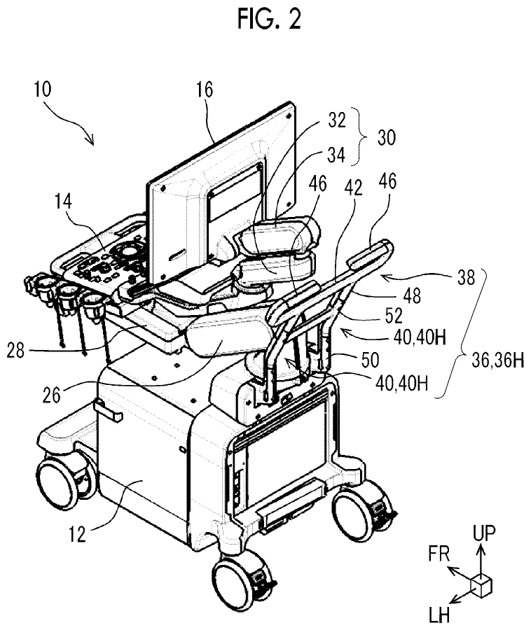

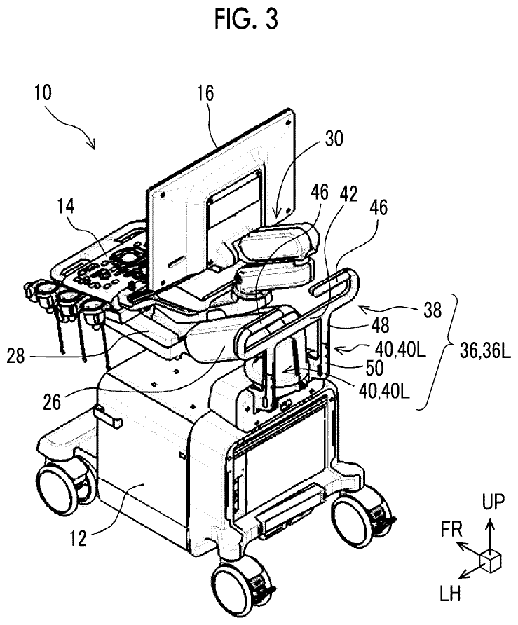

An embodiment of the present invention will be described below with reference to the drawings. FIGS. 1 to 3 are perspective views showing an appearance of an ultrasound diagnostic apparatus 10 related to the present embodiment. FIG. 1 is a perspective view as viewed from a front side, and FIGS. 2 and 3 are perspective views as viewed from a rear side. In the following description, terms indicating a positional relationship, orientations, and directions, such as front, rear, left, right, up, and down, are based on the ultrasound diagnostic apparatus 10, and a side on which an operator operating the ultrasound diagnostic apparatus 10 for diagnosis is present is the front. In each drawing, an arrow FR indicates a front side, an arrow UP indicates an upper side, and an arrow LH indicates a left side.

The ultrasound diagnostic apparatus 10 includes an apparatus body 12, an operation panel 14, a main monitor 16, and a sub monitor 18. The apparatus body 12 has a substantially box shape or a rectangular parallelepiped shape, and accommodates an arithmetic device for performing signal processing, a power supply device, a connection device for inputting and outputting signals, and the like therein. A front leg portion 20 and a rear leg portion 22 are provided at front and rear lower portions of the apparatus body 12 to protrude forward and rearward, and each of the front leg portion 20 and the rear leg portion 22 is provided with two casters 24. A total of four casters 24 are disposed at four corners of the apparatus body 12.

A support arm 26 is installed on the apparatus body 12, and an operation panel base 28 is supported on the support arm 26. The operation panel 14 and the sub monitor 18 are mounted on the operation panel base 28. An articulated arm 30 is installed at a rear end portion of the operation panel base 28, and the main monitor 16 is supported by the articulated arm 30. More specifically, the articulated arm 30 includes two links, that is, a first link 32 and a second link 34 (see FIG. 2), one end of the first link 32 is coupled to the operation panel base 28, one end of the second link 34 is connected to the other end of the first link 32, and the main monitor 16 is supported at the other end of the second link 34. Hereinafter, a state of the articulated arm 30 shown in FIGS. 2 and 3, that is, a state where the first link 32 and the second link 34 are disposed in parallel to overlap with each other and are disposed along a front-rear direction in a plan view will be referred to as a home position of the articulated arm 30.

A push handle 36 is fixed to an upper portion of a rear surface of the apparatus body 12. The push handle 36 includes a handle top 38 that extends in a left-right direction as a whole and handle supports 40 that support the handle top 38. The handle top 38 includes a lateral bar 42 that extends in the left-right direction, curved portions 44 that extend in a U-shape from both ends of the lateral bar 42, and grip portions 46 that further extend from the curved portions 44 and are disposed above the lateral bar 42 in parallel to the lateral bar 42. Ends of the left and right grip portions 46 facing each other are separated from each other.

The two handle supports 40 are installed upright on the apparatus body 12, and the handle top 38, particularly the lateral bar 42 of the handle top 38, is fixed to the upper end of each handle support 40. The two handle supports 40 are disposed at positions separated from each other in the left-right direction.

In the ultrasound diagnostic apparatus 10, a height of the push handle 36, that is, a position of the handle top 38 in an up-down direction can be changed. The handle top 38, particularly the grip portions 46 can be positioned at positions where a tall worker and a short worker easily use the handle top 38, particularly the grip portions 46. FIG. 2 shows a state where a height of the push handle 36 is high, and FIG. 3 shows a state where a height of the push handle 36 is low. In the ultrasound diagnostic apparatus 10, the height of each grip portion 46 of the push handle 36 can be set to 1040 mm and 900 mm.

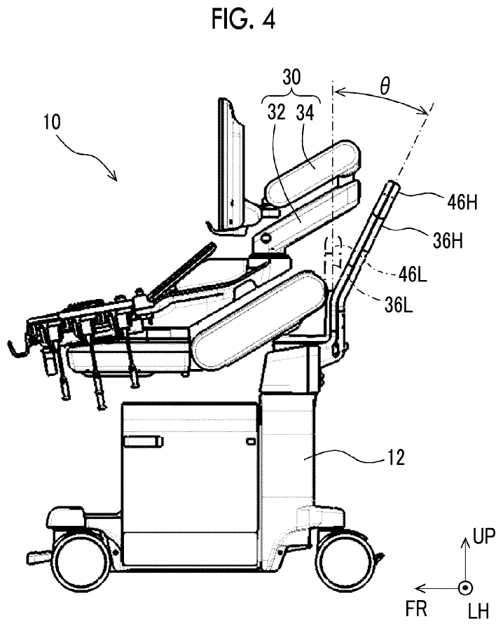

In FIG. 4, the push handles 36 having different heights are shown, a push handle 36H having a high height is shown by a solid line, and a push handle 36L having a low height is shown by a one-dot chain line. In a case where each portion of the push handle 36H having a high height and each portion of the push handle 36L having a low height need to be distinguished from each other in the following description, “H” is added to the reference numeral of each of the portions related to the push handle 36H having a high height and “L” is added to the reference numeral of each of the portions related to the push handle 36L having a low height.

A position of an upper end of the push handle 36H having a high height, that is, a position of each grip portion 46H is higher than a position of an upper end of the push handle 36L having a low height (a position of each grip portion 46L) and is positioned behind the position of the upper end of the push handle 36L having a low height (the position of each grip portion 46L). The push handle 36H is bent, and a lower portion thereof is disposed in a vertical direction whereas an upper portion thereof is inclined rearward. The grip portions 46L at a low position are positioned below the articulated arm 30 positioned at the home position with a space therebetween, and are positioned in front of a rear end of the articulated arm 30. The grip portions 46H at a high position are positioned at a height substantially identical to the height of the rear end of the articulated arm 30 positioned at the home position, but are positioned behind the rear end of the articulated arm 30 with a space therebetween. For this reason, interference between the push handle 36H having a high height and the articulated arm 30 is prevented. A rear inclination angle θ of the push handle 36H having a high height may be 20° to 30°. In the ultrasound diagnostic apparatus 10, the rear inclination angle θ is 26°.

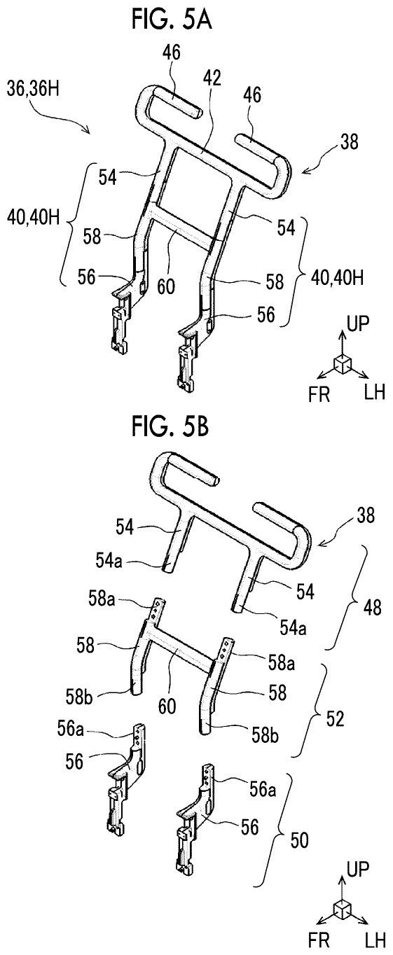

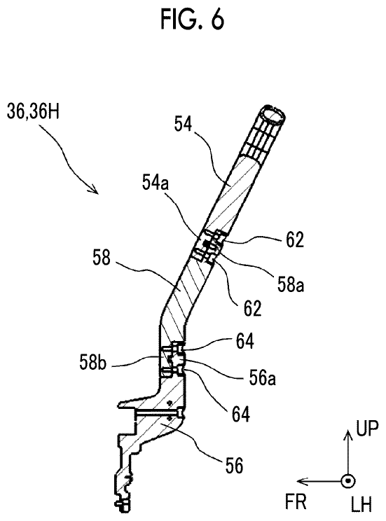

FIGS. 5A and 5B are perspective views showing the push handle 36, in which FIG. 5A shows the push handle 36H having a high height and FIG. 5B is an exploded perspective view of the push handle 36H of which components are disassembled. FIG. 6 is a diagram showing a cross section of the push handle 36H having a high height that includes a center line of one handle support 40.

The push handle 36H having a high height includes an upper handle component 48 that includes the handle top 38, a lower handle component 50 that is fixed to the apparatus body 12, and an intermediate handle component 52 that is interposed between the upper handle component 48 and the lower handle component 50 and connects the upper handle component 48 and the lower handle component 50. The upper handle component 48 and the lower handle component 50 can be directly coupled to each other without the intermediate handle component 52 therebetween, and the push handle 36L having a low height is formed in this case.

Each handle support 40 is divided into three members corresponding to the components 48, 50, and 52 of the push handle 36. A portion of the handle support 40 belonging to the upper handle component 48 is an upper support member 54, a portion of the handle support 40 belonging to the lower handle component 50 is a lower support member 56, and a portion of the handle support 40 belonging to the intermediate handle component 52 is an intermediate support member 58. The upper handle component 48 is integrally formed of the handle top 38 and the two upper support members 54 and has a shape of substantially a Greek letter pi (II). The lower handle component 50 is formed of two independent lower support members 56, and the lower support members 56 are fixed to a push handle fixing portion of the apparatus body 12 by bolts and extend upward in the vertical direction. The intermediate handle component 52 is integrally formed of two bent intermediate support members 58 and a lateral member 60 connecting the two bent intermediate support members 58, and has a substantially H-shape.

At a coupled portion between the upper support member 54 and the intermediate support member 58, end portions of the respective members are provided with halved portions having a half of a cross-sectional shape of the handle support 40 and these halved portions are coupled to each other by bolts. As shown in FIG. 6, a coupling portion 54a of the upper support member 54 and a coupling portion 58a of the intermediate support member 58 are coupled to each other by two bolts 62. In a case where the cross-sectional shape of the handle support 40 is a circular shape, the cross-sectional shapes of the coupling portions 54a and 58a are semicircular shapes. Further, in a case where the cross-sectional shape of the handle support 40 is an elliptical shape, each of the coupling portions 54a and 58a may have a shape of one part of an ellipse that is divided along a minor axis. The cross-sectional shape of the handle support 40 may be other than a circular shape and an elliptical shape, and may be, for example, a shape of a track of an athletic field, which consists of two semicircles facing each other and two straight lines connecting ends of the semicircles facing each other. Furthermore, the cross-sectional shape of the handle support 40 may vary depending on a portion. In this case, it is desirable that the cross-sectional shape smoothly changes so that a step is not formed on the surface of the handle support 40.

A coupled portion between the lower support member 56 and the intermediate support member 58 has a structure identical to the structure of the coupled portion between the upper support member 54 and the intermediate support member 58, and end portions of the respective members are provided with halved portions having a half of the cross-sectional shape of the handle support 40 and these halved portions are coupled to each other by bolts. A coupling portion 56a of the lower support member 56 and a coupling portion 58b of the intermediate support member 58 are coupled to each other by two bolts 64. The handle support 40 including the intermediate support member 58 is a handle support 40H of the push handle 36H having a high height. The handle support 40H is bent at the intermediate support member 58, so that an upper portion of the handle support 40H is inclined rearward.

Since the structure of the coupled portion between the upper support member 54 and the intermediate support member 58 and the structure of the coupled portion between the lower support member 56 and the intermediate support member 58 are identical to each other, the upper support member 54 and the lower support member 56 can be directly coupled to each other. That is, the coupling portion 54a of the upper support member 54 and the coupling portion 56a of the lower support member 56 can be aligned with each other and coupled to each other by the bolts 62 or 64. Accordingly, the handle support 40 in a state where the intermediate support member 58 is removed is a handle support 40L of the push handle 36L having a low height. Since the handle support 40L does not include the bent intermediate support member 58, the handle support 40L extends upward in the vertical direction.

It is possible to select the height of the push handle 36 by selecting whether or not to use the intermediate handle component 52. Further, in a case where the push handle 36 is set to a high state, the handle top 38 is positioned close to the rear side as compared to a case where the push handle 36 is set to a low state. Accordingly, interference between the push handle 36 and the articulated arm 30 supporting the main monitor 16 is prevented.

The push handle 36 includes two handle supports 40 in the ultrasound diagnostic apparatus 10 described above, but may include a handle support integrally formed instead.

What is claimed is:

1. A push handle of an ultrasound diagnostic apparatus that is installed upright on an apparatus body of the ultrasound diagnostic apparatus, the push handle comprising:
a handle top that extends in a left-right direction; and
a handle support of which an upper end is fixed to the handle top and a lower end is fixed to the apparatus body,
wherein the handle support includes
an upper support member that is integrated with the handle top,
a lower support member that is provided on the apparatus body, and
an intermediate member of which an upper end portion is attachably and detachably coupled to the upper support member and a lower end portion is attachably and detachably coupled to the lower support member, and
the upper support member and the lower support member are capable of being directly coupled to each other without the intermediate member therebetween.

2. The push handle of an ultrasound diagnostic apparatus according to claim 1,
wherein the intermediate member is bent, and
in a case where the intermediate member is interposed between the upper support member and the lower support member, the handle top is positioned rearward as compared to a case where the intermediate member is not interposed.

3. The push handle of an ultrasound diagnostic apparatus according to claim 1,
wherein the push handle includes two handle supports that are disposed to be separated from each other in the left-right direction, and a lateral member that connects right and left intermediate members.

4. The push handle of an ultrasound diagnostic apparatus according to claim 1,
wherein coupling portions of the upper support member and the intermediate member and coupling portions of the lower support member and the intermediate member have a halved shape, and
in a case where the upper support member and the intermediate member are coupled to each other and the lower support member and the intermediate member are coupled to each other, an outer shape of the coupling portion and an outer shape of a portion adjacent to the coupling portion are identical to each other.
